(19) United States
(12) Patent Application Publication
(10) Pub. No.: US 2006/0147502 A1
(43) Pub. Date: Jul. 6, 2006

(54) Methods for controlling microbial pathogens on currency and mail

(75) Inventors: David William Koenig, Menasha, WI (US); John Edward Laabs, Hortonville, WI (US)

Correspondence Address: SENNIGER POWERS, ONE METROPOLITAN SQUARE, 16TH FLOOR, ST LOUIS, MO, 63102, US

(73) Assignee: Kimberly-Clark Worldwide, Inc., Neenah, WI (US)

(21) Appl. No.: 11/026,289

(22) Filed: Dec. 30, 2004

(51) Int. Cl. A61K 9/70

(52) U.S. Cl. 424/443

(57) ABSTRACT

The present invention relates to methods for minimizing the presence of microbial pathogens on currency or on mail. More particularly, the presence of microbial pathogens may be minimized by introducing an antimicrobial product comprising a base substrate and a volatile and/or non-volatile antimicrobial agent into a money container comprising currency or into an envelope. The antimicrobial agent may be either transferred directly to the currency or envelope by contacting the antimicrobial product with the currency or envelope, or through permeation of the volatile antimicrobial throughout the money container or envelope.

### BACKGROUND OF INVENTION

The present invention generally relates to methods for minimizing the presence of microbial pathogens on currency and mail. More particularly, the presence of microbial pathogens on currency and/or mail is minimized by introducing an antimicrobial product comprising a base substrate and an antimicrobial agent into a money container comprising currency or into an envelope. The antimicrobial agent may be either transferred directly to the currency (or envelope) by contacting the antimicrobial product with the currency or envelope or, when the antimicrobial agent is a volatile antimicrobial, may be contacted with the currency or envelope through permeation of the volatile antimicrobial throughout the money container or envelope. Once the antimicrobial agent has contacted the currency or envelope, the antimicrobial agent may kill or reduce the growth of microbial pathogens present on the currency or envelope, and thus reduce the potential for transfer of the microbial pathogens.

Paper currency often becomes contaminated with microbial pathogens from being widely handled and exchanged for goods and services. In fact, studies have shown that around 87% of paper currency may be contaminated with bacteria that could cause significant infections. Consequently, paper currency may be a vector for the spread of disease. People with stressed or compromised immune systems, including children and the elderly, may be particularly at risk. Furthermore, the possibility of deliberate contamination of paper money with microbial pathogens as a form of biological warfare or terrorist activity has caused concern among public health officials. The removal or minimization of microbial pathogens on paper currency would thus be a significant benefit to public health and safety.

### SUMMARY OF THE INVENTION

The present invention relates to methods for minimizing the presence of microbial pathogens on currency or on mail. More particularly, the present invention relates to minimizing the presence of microbial pathogens on currency or on mail by introducing an antimicrobial product comprising a base substrate and a volatile and/or non-volatile antimicrobial agent into a money container containing currency or into an envelope. When the antimicrobial agent contacts the currency or mail, the amount of active microbial pathogen is reduced resulting in a safer product.

In one aspect, the present invention provides a method for minimizing the presence of microbial pathogens on currency. The method comprises introducing an antimicrobial product into a money container containing currency, wherein the volatile antimicrobial agent is capable of contacting the currency. The antimicrobial product may comprise a base substrate and a volatile antimicrobial agent.

Also provided is a method for minimizing the presence of microbial pathogens on currency. The method comprises introducing an antimicrobial product into a money container containing currency so that the antimicrobial product contacts the currency. The antimicrobial product may comprise a base substrate and a non-volatile antimicrobial agent.

In another aspect, the present invention provides a method for minimizing the presence of microbial pathogens on an envelope. The method comprises introducing an antimicrobial product into the envelope, wherein the volatile antimicrobial agent is capable of contacting the envelope. The antimicrobial product may comprise a base substrate and a volatile antimicrobial agent.

### DETAILED DESCRIPTION OF THE PREFERRED EMBODIMENTS

In accordance with the present invention, it has been discovered that the presence of microbial pathogens on currency or mail may be minimized by introducing an antimicrobial product comprising a base substrate and an antimicrobial agent into a money container comprising currency (or into an envelope). The antimicrobial agent may either be transferred directly to the currency (or envelope) by contacting the antimicrobial product with the currency (or envelope), or may be contacted with the currency (or envelope) through permeation. For example, in one embodiment, the antimicrobial product comprises a volatile antimicrobial agent. The antimicrobial product comprising the volatile antimicrobial agent may be introduced into a money container comprising currency. As the volatile antimicrobial agent is released from the antimicrobial product, the volatile antimicrobial agent permeates the money container, including the paper currency present in the container. As the paper currency is permeated, the volatile antimicrobial comes in contact with microbial pathogens present on the currency, and may act to kill or reduce the growth of the pathogens. Alternately or in addition, the antimicrobial product may comprise a non-volatile (or volatile) antimicrobial agent, and this antimicrobial agent may be transferred to the currency by directly contacting the currency with the antimicrobial product.

Although discussed primarily in terms of currency such as paper currency or currency in the form of coins, the methods described herein are also suitable for use in minimizing the presence of microbial pathogens on other paper products, including mail. For example, an antimicrobial product, as described herein, may be introduced into an envelope before mailing. In one embodiment, the antimicrobial agent is a volatile antimicrobial agent. As the volatile antimicrobial agent is released from the product, the volatile antimicrobial agent permeates the envelope, thus killing or reducing the growth of microbial pathogens that are present on or that may otherwise be transferred to the envelope as it travels through the mail. The antimicrobial agent (whether volatile or non-volatile) may also be transferred directly to the envelope by contacting the envelope with the antimicrobial product. Additionally, it is contemplated that the processes described herein could be used to sanitize multiple envelopes or packages by introducing an antimicrobial product into a recepticle for mail and allowing the antimicrobial product to sanitize the mail.

The term “money container” as used herein is meant to refer to a device suited for holding currency. Such containers include, but are not limited to a wallet, a purse, a drawer of a cash register, a currency transport bag, a money pouch, a pocket, and a cash box, among others.

The term “currency” as used herein is meant to refer to money in any form that is capable of being used as a medium of exchange, including paper currency and coins, bonds, etc. Although discussed primarily in terms of paper currency, the methods described herein may also be used to minimize the presence of microbial pathogens on coins present in the money container. For example, the antimicrobial agent (whether volatile or non-volatile) may be transferred to coins in the money container by directly contacting the coins with the antimicrobial product. The exposed surface of the coins may also be contacted with an antimicrobial agent when a volatile antimicrobial agent permeates the money container.

The term “microbial pathogen” as used herein is meant to refer to any microorganism capable of causing infection or disease. Microbial pathogens include, but are not limited to pathogenic bacteria, viruses, and fungi, such as Staphylococcus aureus, Escherichia coli, Vibrio, Salmonella, Bacillus, Fusarium, and spores of various organisms, among others.

The antimicrobial products for use in the present invention comprise a base substrate. Base substrates suitable for use in the antimicrobial products of the present invention can be made from various materials and fibers. The base substrate can be made from pulp fibers, or other natural fibers, cellulose fibers, synthetic fibers such as polypropylene or polylactic acid, and the like.

Optionally, the antimicrobial product may assume a variety of shapes, including but not limited to, generally circular, oval, square, rectangular, or irregularly shaped depending upon numerous factors. The size of the antimicrobial product may also vary depending upon the desired end use of the product. For example, the base substrate may be sized and shaped to fit into various types of money containers. Although size is not critical, in one embodiment, the substrate is the size and shape of a paper bill. The substrate may also be sized to fit into a compartment of a drawer of a cash register, cash box or currency bag, or may be sized to fit into various shapes and sizes of envelopes.

One desirable base substrate is a tissue product substrate. The present invention is useful with tissue products and tissue paper in general, including but not limited to conventionally felt-pressed tissue paper, high bulk pattern densified tissue paper, and high bulk, uncompacted tissue paper. The tissue paper can be of a homogenous or multi-layered construction, and tissue paper products made therefrom can be of a single-ply or multi-ply construction. The tissue paper desirably has a basis weight of between about 10 g/m² and about 65 g/m², and a density of about 0.6 g/cc or less. More desirably, the basis weight will be about 40 g/m² or less and the density will be about 0.3 g/cc or less. Most desirably, the density will be between about 0.04 g/cc and about 0.2 g/cc. Unless otherwise specified, all amounts and weights relative to the paper are on a dry basis. Stretch in the machine direction can be in the range of from about 5% to about 20%. Stretch in the cross-machine direction can be in the range of from about 3% to about 20%. Tensile strengths in the machine direction can be in the range of from about 100 to about 5,000 grams per inch of width. Tensile strengths in the cross-machine direction are in the range of from about 50 grams to about 2,500 grams per inch of width.

Conventionally pressed tissue paper and methods for making such paper are well known in the art. For example, high bulk pattern densified tissue paper suitable for use in the present invention is disclosed in U.S. Pat. No. 3,301,746 (Sanford et al.), issued Jan. 31, 1967; U.S. Pat. No. 3,974,025 (Ayers), issued Aug. 10, 1976; and U.S. Pat. No. 4,191,609 (Trokhan), issued Mar. 4, 1980; and U.S. Pat. No. 4,637,859 (Trokhan), issued Jan. 20, 1987; all of which are incorporated by reference. Additionally, uncompacted, nonpattern-densified tissue paper structures suitable for use in the present invention are described in U.S. Pat. No. 3,812,000 (Salvucci et al.), issued May 21, 1974 and U.S. Pat. No. 4,208,459 (Becker et al.), issued Jun. 17, 1980, both of which are incorporated by reference.

Such paper is typically made by depositing a papermaking furnish on a foraminous forming wire, often referred to in the art as a Fourdrinier wire. Once the furnish is deposited on the forming wire, it is referred to as a web. The web is dewatered by pressing the web and drying at an elevated temperature. The particular techniques and typical equipment for making webs according to the process just described are well known to those skilled in the art. In a typical process, a low consistency pulp furnish is provided from a pressurized headbox, which has an opening for delivering a thin deposit of pulp furnish onto the Fourdrinier wire to form a wet web. The web is then typically dewatered to a fiber consistency of between about 7% and about 25% (total web weight basis) by vacuum dewatering and further dried by pressing operations wherein the web is subjected to pressure developed by opposing mechanical members, for example, cylindrical rolls. The dewatered web is then further pressed and dried by a steam drum apparatus known in the art as a Yankee dryer. Pressure can be developed at the Yankee dryer by mechanical means such as an opposing cylindrical drum pressing against the web. Multiple Yankee dryer drums can be employed, whereby additional pressing is optionally incurred between the drums. The formed sheets are considered to be compacted since the entire web is subjected to substantial mechanical compressional forces while the fibers are moist and are then dried while in a compressed state.

The papermaking fibers utilized in preparing tissue paper for the products of the present invention will normally include fibers derived from wood pulp. Other cellulosic fibrous pulp fibers, such as cotton linters, bagasse, etc., can be utilized and are intended to be within the scope of this invention. Synthetic fibers, such as rayon, polyethylene and polypropylene fibers, can also be utilized in combination with natural cellulosic fibers. One exemplary polyethylene fiber that can be utilized is Pulpex.RTM., available from Hercules, Inc. (Wilmington, Del.).

Applicable wood pulps include chemical pulps, such as Kraft, sulfite, and sulfate pulps, as well as mechanical pulps including, for example, groundwood, thermo-mechanical pulp and chemically modified thermo-mechanical pulp. Pulps derived from both deciduous trees and coniferous trees can be utilized. Also useful in the present invention are fibers derived from recycled paper, which can contain any or all of the above categories as well as other non-fibrous materials such as fillers and adhesives used to facilitate the original papermaking.

In addition to papermaking fibers, the papermaking furnish used to make tissue paper structures can have other components or materials added thereto as can be or later become known in the art. The types of additives desirable may include certain dry strength and lint control additives known in the art, such as starch binders. In addition to reducing tinting of the finished tissue paper product, low levels of starch binders also impart a modest improvement in the dry tensile strength without imparting stiffness that could result from the addition of high levels of starch. Typically, the starch binder is included in an amount such that it is retained at a level of from about 0.01 to about 2%, preferably from about 0.1 to about 1%, by weight of the dry tissue paper.

Other materials suitable for use as the base substrate of the antimicrobial product are well known to those skilled in the art, and may include a fibrous sheet material, which may be either woven or nonwoven. For example, the antimicrobial products described herein may include nonwoven fibrous sheet materials, which include meltblown, coform, air-laid, bonded-carded web materials, hydroentangled materials, spun-bound materials, and the like, and combinations thereof. Such materials can be comprised of synthetic or natural fibers, or a combination thereof. Examples of natural fibers suitable for use in the present invention include cellulosic fibers such as wood pulp fibers, cotton fibers, flax fibers, jute fibers, silk fibers and the like. Examples of thermoplastic polymeric fibers suitable for use with the present invention include polyolefins such as polypropylene and polyethylene, polyamides, and polyesters such as polyethylene teraphthalate. Alternative synthetic fibers which may be suitable include staple nylon and rayon fibers. The layer or layers of the antimicrobial prouduct can be woven or nonwoven materials.

The materials may be formed into a single or multi-layered base substrate, and may be varied to provide different physical properties, such as softness, resiliency, strength, flexibility, integrity, toughness, thickness, tear resistance, surface texture, and the like, and combinations thereof. The base substrate can be configured to provide all desired physical properties within one layer, or configured to provide only specific physical properties within individual layers of a multi-layered product.

If one or more layers of the base substrate is a combination of polymeric and natural fibers, such as polypropylene and cellulosic fibers, the relative percentages of the polymeric fibers and natural fibers in the layer can vary over a wide range depending on the desired characteristics of the antimicrobial product. For example, the layer may comprise from about 20 to about 100 weight percent, desirably from about 20 to about 60 weight percent, and more desirably from about 30 to about 40 weight percent of polymeric fibers based on the dry weight of the layer. Such a layer of polymeric and natural fibers may be manufactured by any method known to those skilled in the art.

Generally, it is desirable that a layer comprising both polymeric and natural fibers be formed by a coform process for a more uniform distribution of the polymeric and natural fibers within the layer. Such coform layers are manufactured generally as described in U.S. Pat. No. 4,100,324 to Anderson et al. which issued Jul. 11, 1978; U.S. Pat. No. 4,604,313 to McFarland et al. which issued Aug. 5, 1986; and U.S. Pat. No. 5,350,624 which issued Sep. 27, 1994; which is herein incorporated by reference to the extent they are consistent herewith.

Typically, such coform layers comprise a gas-formed matrix of thermoplastic polymeric meltblown microfibers, such as, for example, polypropylene microfibers, and cellulosic fibers, such as, for example, wood pulp fibers. A coform layer is formed by initially forming at least one primary air stream containing the synthetic or polymeric fibers and merging the primary stream with at least one secondary stream of natural or cellulosic fibers. The primary and secondary streams are merged under turbulent conditions to form an integrated stream containing a thorough, homogeneous distribution of the different fibers. The integrated air stream is directed onto a forming surface to air form the layer of material. A multiplicity of these coform layers can then be formed in succession to provide a web of multiple coform layers.

The different fibers in the different layers of the layered base substrate of the present invention, such as the polypropylene and polyethylene microfibers set forth above, typically may not be compatible with and may not bond to each other. However, the different fibers may entangle with each other resulting in suitable securement between the layers. For example, in a layered base substrate containing a coform layer of polyethylene and cellulosic fibers and a coform layer of polypropylene and cellulosic fibers, the polyethylene and polypropylene fibers may entangle with each other and the cellulosic fibers and may at least partially bond to the cellulosic fibers which results in securement between the layers. Such interlayer bonding and entanglement may be enhanced by a thermo-mechanical process wherein the layered base substrate is passed between a heated smooth anvil roll and a heated pattern roll. The pattern roll may have any raised pattern which provides the desired entanglement and interlayer bonding. Desirably, the pattern roll defines a raised pattern which defines a plurality of bond locations which define a bond area of between about 4 and about 30 percent of the total area of the roll for improved interlayer attachment.

The base substrate for the antimicrobial product may have a total basis weight of from about 25 to about 120 grams per square meter and desirably from about 40 to about 90 grams per square meter. The basis weight of the base substrate may vary depending upon one or more desired characteristics of the antimicrobial product. For example, a suitable base substrate may define a basis weight of from about 60 to about 80 grams per square meter and desirably about 75 grams per square meter. In a particular embodiment wherein the base substrate includes coform layers of polypropylene and cellulosic fibers and polyethylene and cellulosic fibers, the layered base substrate defines a basis weight of from about 60 to about 90 grams per square meter and desirably about 80 grams per square meter.

In a particular embodiment, the base substrate for the antimicrobial product may define a tensile strength of at least about 1.23 Newtons per centimeter in the machine direction and at least about 0.70 Newtons per centimeter in the cross machine direction. Antimicrobial products having alternate ranges of tensile strength may also be effectively employed. As used herein, the term “machine direction” refers to the direction in which the material is manufactured while the cross machine direction refers to a direction which is perpendicular to the machine direction.

In a particular embodiment, wherein the base substrate includes coform layers of polypropylene and cellulosic fibers and polyethylene and cellulosic fibers, the layered base substrate defines a tensile strength of from about 1.31 to about 3.50 Newtons per centimeter in the machine direction and from about 0.84 to about 1.40 Newtons per centimeter in the cross machine direction, and desirably from about 1.58 to about 1.93 Newtons per centimeter in the machine direction and from about 0.93 to about 1.11 Newtons per centimeter in the cross machine direction. In such a configuration, the coform layer, which includes polypropylene fibers, provides the majority of the strength to the base substrate.

Alternatively, the base substrate incorporating the antimicrobial agents described herein can comprise a composite, which includes multiple layers of materials such as those described in U.S. Pat. No. 6,028,018, which is incorporated by reference. For example, the base substrate may include a three layer composite, which includes an elastomeric film or meltblown layer between two coform layers as described above. In such a configuration, the coform layers may define a basis weight of from about 15 to about 30 grams per square meter and the elastomeric layer may include a film material such as a polyethylene metallocene film.

As discussed above, one or more antimicrobial agents may be introduced into or onto the base substrate to form an antimicrobial product suitable for sanitizing currency. The antimicrobial product may then be used in the methods of the present invention to minimize the presence of microbial pathogens on currency or mail.

As used herein, the term “antimicrobial agent” is meant to include any agent capable of killing a microbial pathogen or reducing the growth of a microbial pathogen, thus mimimizing the presence of the microbial pathogen on currency, mail, envelopes, or other desirable products. Suitable antimicrobial agents include both volatile and non-volatile antimicrobial agents. As discussed above, the antimicrobial agent may either be transferred directly to the currency (or mail, envelope, etc.), and/or may be contacted with the currency (or mail, envelope, etc.) through permeation.

Any amount of antimicrobial agent capable of minimizing the presence of microbial pathogens on the currency (or mail, envelope, etc.) may be introduced into or onto the base substrate of the antimicrobial product. In one embodiment, the base substrate comprises from about 0.1% (by weight of the base substrate) to about 5% (by weight of the base substrate) of the antimicrobial agent. Preferably, the base substrate comprises from about 1% (by weight of the base substrate) to about 2% (by weight of the base substrate) of the antimicrobial agent. In addition, the base substrate may comprise one or more non-volatile antimicrobial agent, one or more volatile antimicrobial agent, or combinations thereof.

In one embodiment, the antimicrobial agent comprises one or more non-volatile antimicrobial agents. Numerous examples of non-volatile antimicrobial agents are known in the art and are suitable for use in the methods described herein. For example, a desirable non-volatile antimicrobial agent comprises a mixture of organic acids, such as benzoic acid and poly(hexamethylene biguanide hydrochlorice) (i.e., PHMB). Without wishing to be bound to any particular theory, it is believed that when the antimicrobial product comprising the non-volatile antimicrobial agent contacts currency (or an envelope, mail, etc.), the antimicrobial agent is transferred to the surface of the currency, thereby killing or reducing the growth of any microbial pathogens that may be present on the currency (or envelope, mail, etc.). Volatile antimicrobial agents may be transferred to the surface of the currency in a similar manner.

Other suitable examples of non-volatile antimicrobial agents include Yucca species extracts such as Yucca schidigera and more particularly a Yucca schidigera solution sold under the trade designation Yucca 70 by Sher-Mar Enterprises of Poway, Calif. The non-volatile antimicrobial agent may also be a broad spectrum antimicrobial agent, including synthetic antimicrobial agents or naturally occurring antimicrobial agents such as a botanical extract, herb, or essential oil.

Suitable synthetic-type broad spectrum antimicrobial agents include, for example, alcohols having from one to about 6 or 7 carbon atoms per molecule. Alcohols exhibit antimicrobial properties when used at sufficiently high concentrations. Other suitable synthetic-type broad spectrum antimicrobial agents include triclosan (2,4,4′-trichloro-2′-hydroxydiphenyl ether), triclocarban, p-chloro-m-xylenol, benzalkonium chloride, chlorohexidine gluconate, hexachlorophene and the like, and combinations thereof.

Suitable natural broad spectrum antimicrobial agents include, for example, aloe vera, folic acid, calendula flower, echinacea purpurea tops, gota kola extract, chlorophyll, phytoplenolin extract, chamomile flower, blood root, prickly ash bark, green tea leaf, oregano leaf, peppermint oil, cinnamon bark, eucalyptus leaf, lavender oil, bio-saponin concentrate, olive leaf extract, black walnut green hulls, clove leaf, thyme herb, grapefruit seed extract, vegetable glycerin, and combinations thereof.

In another embodiment, the antimicrobial agent comprises one or more volatile antimicrobial agent. Without wishing to be bound to any particular theory, it is believed that volatile antimicrobial agents present on the antimicrobial products vaporize off the product and permeate throughout the entire money container (or mail, envelope, etc.), including any paper currency present in the container. As the currency is permeated (or the surface is contacted in the case of coins), the volatile antimicrobial agent comes in contact with any microbial pathogens present on the currency and acts to kill or reduce the growth of the pathogens. In this way, the presence of microbial pathogens present on currency in the money container is minimized.

Suitable volatile antimicrobial agents are known in the art and include, among others, phenothiazinium dyes, such as methylene blue, dimethyl methylene blue, and toluidine blue, among others; peroxides, such as hydrogen peroxide, mannitol peroxide, and urea peroxide; S-nitrosgluthathione; nitric oxide; nitric oxide donors; ozone; hypochlorous acid; chlorine dioxide; dimethyl fumarate; essential oils of Thymus, Ferulago, Foeniculum vulgare, Crithmum maritimum, and tea tree; allylsulfide constituents of garlic volatile oil and Calamintha nepeta; various other reactive oxygen species; and combinations thereof. Particularly preferred volatile antimicrobial agents include chlorine dioxide, the peroxides, and essential oils of Thymus, Ferulago, Foeniculum vulgare, Crithmum maritimum, and tea tree. Optionally, the volatile antimicrobial agent may be one that imparts a pleasing or fresh scent when vaporized off of the product. Examples of such volatile antimicrobials include rose oil, lemon grass oil, and banana oil (1-butanol, 3-methyl acetate).

Because of their volatile nature, it is preferable to provide a means of protecting the volatile antimicrobial agent from exposure to the environment during shipping and handling of the antimicrobial product. Stated in another way, it is generally preferred to delay activation of the product until use. Failure to do this may result in the vaporization of the volatile antimicrobial from the product prior to introduction of the product into the money container or envelope, and a loss of antimicrobial benefit.

Therefore, in one embodiment of the present invention, the antimicrobial product may comprise volatile antimicrobial agents (or non-volatile antimicrobial agents) which are encapsulated or entrapped within a thin layer of material. The volatile antimicrobial agent can be encapsulated in a number of shell-like materials including, for example, cellulose-based polymeric materials (i.e., ethyl cellulose), carbohydrate-based materials (e.g., cationic starches and sugars), polyglycolic acid, polylactic acid, and lactic acid-based aliphatic polyesters, and materials derived therefrom (e.g., dextrins and cyclodextrins). Other examples of suitable shell-like materials include liposomes, nanosomes, nanoparticles, collagen, gelatin, melamine resin, silicon resin, and combinations thereof. Alternatively, the encapsulating material may comprise a natural or synthetic polymer system (microsponge) such as, for example, acrylate polymers, acrylate copolymers, starch, silica, oat, and combinations thereof.

Encapsulating the volatile antimicrobial agent can protect the volatile antimicrobial agent from exposure to harsh environmental conditions which may result in premature oxidation, vaporization, and degradation of the volatile antimicrobial and/or the overall antimicrobial product. Encapsulation also allows for the separation of any incompatible components within the product, which allows greater flexibility in the components which can be used to make the product. Additionally, encapsulation allows for a controlled release of the volatile antimicrobial agents during use of the antimicrobial product. A controlled release may include a triggered release, sustained release, or a combination of these release mechanisms, wherein the active ingredient is released from the encapsulant by a number of mechanisms including, for example, pressure, ultraviolet light, capillary forces, and wetting with water, or is gradually released as the encapsulant breaks down over time. In a similar manner, non-volatile antimicrobial agents may also be encapsulated.

Typically, the particle size of the microencapsulated materials and the polymeric entrapment materials are from about 0.1 micrometers to about 40 micrometers, desirably from about 0.3 micrometers to about 20 micrometers, and still more desirably from about 0.5 micrometers to about 15 micrometers.

The microencapsulation shell thickness may vary depending upon the volatile (or non-volatile) antimicrobial agent utilized, and is generally manufactured to allow the encapsulated agent to be covered by a thin layer of encapsulation material, which may be a monolayer or thicker laminate layer, or may be a composite layer. The microencapsulation layer should be thick enough to resist cracking or breaking of the shell during handling or shipping of the product. The microencapsulation layer should also be constructed such that humidity from atmospheric conditions during storage, shipment, or wear will not cause a breakdown of the microencapsulation layer and result in a premature release of the volatile antimicrobial agent.

In one embodiment, precursors of a volatile antimicrobial agent are encapsulated. The phrase “precursors of a volatile antimicrobial agent” as used herein is meant to include compounds or agents that when contacted with another suitable compound or agent will form a volatile antimicrobial agent. Upon rupture of the encapsulant, the precursors may come in contact and form a volatile antimicrobial agent, which may then permeate the money container or envelope, as described herein. Any of the volatile antimicrobial agents described herien may be formed in this manner. This method is particularly desirable when the volatile antimicrobial agent is chlorine dioxide. In a similar manner, photo activated dyes, such as the phenothiazinium dyes described herein, may be encapsulated. Without wishing to be bound to any particular theory, it is believed that upon exposure to light, preferably red light, the encapsulant may be broken and the dye activated. Stated another way, upon exposure to light the dye may be released from the encapsulant, become volatile, and permeate throughout the money container or envelope, as discussed above. In light of their low toxicity, these dyes may advantageously be used in the methods described herein.

The antimicrobial agents described herein (whether encapsulated or non-encapsulated) may be introduced into or onto the base substrate by any suitable means known in the art, including impregnating, spraying, soaking, and printing, among others. In one embodiment, when the antimicrobial product is a sheet comprising a top and a bottom side, the antimicrobial agents may be introduced onto both sides of the product. Alternately, the antimicrobial agents are introduced onto only one side of the antimicrobial product. Optionally, when the antimicrobial agents are present on only one side of the antimicrobial product, the other side of the antimicrobial product is sealed to prevent the antimicrobial agents from migrating through the product and away from the product surface (or in the case of volatile antimicrobial agents from being volatilized by exposure to the environment through the non-sealed side of the product). In a desirable embodiment, the antimicrobial agents are held at or near the surface of the antimicrobial product, so as to increase the contact between the antimicrobial agent and microbial pathogens.

In one embodiment, the volatile or non-volatile antimicrobial agents present on the antimicrobial product may be covered by a removable protective covering to protect the antimicrobial agents from exposure to environmental conditions which may result in premature oxidation and degradation of the antimicrobial agents and the overall product, or in the case of volatile antimicrobial agents, premature vaporization of the volatile antimicrobial agent. One or optionally both sides of the antimicrobial product may be covered by a removable protective covering. Prior to inserting the antimicrobial product into the money container, the protective coating is preferably removed, for example, by lifting the edge of the covering and peeling the covering from the antimicrobial product. Upon removal of the protective covering, the antimicrobial agents on the product are exposed to the environment and may, in the case of volatile antimicrobial agents, vaporize and permeate the money container, as described herein. The methods of the present invention may thus further comprise removing the protective covering prior to introducing the antimicrobial product into the container.

Alternately, the antimicrobial product may be contained within a sealable pouch. Like the removable protective covering, described above, the sealable pouch may act to protect the antimicrobial agents present in or on the antimicrobial product from exposure to environmental conditions which may result in premature oxidation and degradation of the antimicrobial agents and the overall product, or the premature vaporization of volatile antimicrobial agents. Prior to inserting the antimicrobial product into the money container or envelope, the sealable pouch is preferably opened and the antimicrobial product removed. Upon removal from the sealable pouch, the antimicrobial agents on the product are exposed to the environment, as described above. Optionally, the sealable pouch may contain more than one antimicrobial product, and may be resealed after each antimicrobial product is removed to protect the remaining antimicrobial products from exposure to the environment.

In addition to or as an alternative to the use of an antimicrobial agent, the presence of microbial pathogens on currency or envelopes may be minimized by generating a small electric field that contacts the currency (or mail, envelope, etc.). Without wishing to be bound to any particular theory, it is believed that as the electric field passes through the currency or envelope, the current disrupts or disorganizes cellular membranes of the microbial pathogens. Depending on the strength of the electric field, the microbial pathogens may be killed. Alternately, the disruption of the cellular membrane may be used in combination with an antimicrobial agent, wherein the disruption enhances the ability of the antimicrobial agent to penetrate the cellular membrane of the microbial pathogen, thus enhancing the antimicrobial agent's ability to kill or reduce the growth of the microbe.

Therefore, in one embodiment, the antimicrobial product comprises a current source trapped into the base substrate. Suitable current sources are known in the art and may include, for example, micro-polymer batteries. Preferably, the current source is capable of producing an amperage of from about 10 μA to about 200 μA, more preferably from about 50 μA to about 100 μA. The placement of the poles of the current source within the base substrate is not critical. However, it is generally preferred that the poles of the current source be placed on opposite ends of the base substrate to maximize the size of the electric field that is produced. The current may then pass through a bill or stack of bills that is in contact with the base substrate.

Thus in one embodiment, the present invention provides a method for minimizing the presence of microbial pathogens on currency, the method comprising introducing an antimicrobial product into a money container containing currency. The antimicrobial product preferably may comprise a base substrate and a current source, the current source capable of producing an amperage of from about 10 μA to about 200 μA. Preferably the antimicrobial product is in contact with the currency. The antimicrobial product may comprise, in addition to the current source, an antimicrobial agent, as described herein. Similar methods may be used to minimize the presence of microbial pathogens on mail, envelopes, or other paper products.

As various changes could be made in the above methods without departing from the scope of the invention, it is intended that all matter contained in the above description shall be interpreted as illustrative and not in a limiting sense.

What is claimed is:

1. A method for minimizing the presence of microbial pathogens on currency, the method comprising introducing an antimicrobial product into a money container containing currency, said antimicrobial product comprising a base substrate and a volatile antimicrobial agent, wherein the volatile antimicrobial agent is capable of contacting the currency.

2. The method of claim 1 wherein said base substrate comprises a material selected from the group consisting of cellulosic fibers and synthetic fibers.

3. The method of claim 1 wherein said base substrate is a woven material.

4. The method of claim 1 wherein said base substrate is a non-woven material.

5. The method of claim 1 wherein said base substrate is impregnated with said volatile antimicrobial agent.

6. The method of claim 1 wherein said base substrate comprises a top side and a bottom side, said top side and said bottom side comprising the volatile antimicrobial agent.

7. The method of claim 1 wherein said base substrate comprises a top side and a bottom side, and wherein only one side of the base substrate comprises the volatile antimicrobial agent.

8. The method of claim 1 wherein said base substrate comprises from about 0.1% (by weight of the base substrate) to about 5% (by weight of the base substrate) of the volatile antimicrobial agent.

9. The method of claim 8 wherein said base substrate comprises from about 1% (by weight of the base substrate) to about 2% (by weight of the base substrate) of the volatile antimicrobial agent.

10. The method of claim 1 wherein said volatile antimicrobial agent is selected from the group consisting of phenothiazinium dyes and peroxides.

11. The method of claim 10 wherein said peroxides are selected from the group consisting of hydrogen peroxide, mannitol peroxide, urea peroxide, and combinations thereof.

12. The method of claim 10 wherein the phenothiazinium dyes are selected from the group consisting of methylene blue, dimethyl methylene blue, toluidine blue, and combinations thereof.

13. The method of claim 1 wherein said volatile antimicrobial agent is selected from the group consisting of S-nitrosogluthathione; nitric oxide; hypochlorous acid; chlorine dioxide; dimethyl fumarate; essential oils of Thymus, Ferulago, Foeniculum vulgare, Crithmum maritimum, and tea tree; allylsulfide constituents of garlic volatile oil, and Calamintha nepeta; and combinations thereof.

14. The method of claim 13 wherein said volatile antimicrobial agent is chlorine dioxide.

15. The method of claim 1 wherein said volatile antimicrobial agent is encapsulated in a shell material.

16. The method of claim 15 wherein the shell material comprises a material selected from the group consisting of cellulose-based polymeric materials, carbohydrate-based materials and materials derived therefrom.

17. The method of claim 1 wherein said antimicrobial product further comprises a removable protective covering, wherein said removable protective covering is removed from said antimicrobial product prior to introducing the antimicrobial product into said money container.

18. The method of claim 1 wherein said currency is selected from the group consisting of paper currency, coins, and combinations thereof.

19. The method of claim 1 wherein said antimicrobial product further comprises a current source embedded in said base substrate, said current source being capable of producing an amperage of from about 10 μA to about 200 μA.

20. A method for minimizing the presence of microbial pathogens on currency, the method comprising introducing an antimicrobial product into a money container containing currency so that the antimicrobial product contacts the currency, said antimicrobial product comprising a base substrate and a non-volatile antimicrobial agent.

21. The method of claim 20 wherein said non-volatile antimicrobial agent comprises PHMB.

22. The method of claim 20 wherein said currency is selected from the group consisting of paper currency, coins, and combinations thereof.

23. A method for minimizing the presence of microbial pathogens on an envelope, the method comprising introducing an antimicrobial product into the envelope, said antimicrobial product comprising a base substrate and a volatile antimicrobial agent, wherein the volatile antimicrobial agent is capable of contacting the envelope.
